(19) United States
(12) Plant Patent Application Publication
(10) Pub. No.: US 2014/0331370 P1
(43) Pub. Date: Nov. 6, 2014

(54) Westringia fruticosa plant named 'WES02'

(50) ;

(71) Applicant: Graham Brown, Cobbity (AU)

(72) Inventor: Graham Brown, Cobbity (AU)

(21) Appl. No.: 13/986,462

(22) Filed: May 6, 2013

(51) Int. Cl. A01H 5/00

(52) U.S. Cl. PLT/226

(57) ABSTRACT

‘WES02’ is a distinctive variety of Westringia fruticosa which is characterized by a combination of a rotund, or ball-shaped, growth habit, short plant height and narrow, fine-textured foliage.

### LATIN NAME OF THE GENUS AND SPECIES

The Latin name of the genus and species of the novel variety disclosed herein is Westringia fruticosa.

### VARIETY DENOMINATION

The inventive variety of Westringia fruticosa disclosed herein has been given the variety denomination ‘WES02’.

### BACKGROUND OF THE INVENTION

The present invention relates to a new and distinct perennial variety of Westringia fruticosa, which has been given the variety denomination of ‘WES02’. Its market class is that of a shrub. ‘WES02’ is intended for use in general landscaping and as a decorative plant.

Parentage: The Westringia fruticosa variety ‘WES02’ is the result of a controlled pollination breeding program from 2004 to 2009 in Cobbitty, NSW Australia. ‘WES02’ is a progeny of said breeding program which resulted from the cross pollination of male parent line Westringia fruticosa ‘x2003.5.7’ (unpatented) and female parent line Westringia fruticosa ‘x2003.5.1’ (unpatented). Controlled pollination of the parents occurred in September of 2004 and resulting seed from said cross was sown in March of 2005 in accord with standard greenhouse propagation practices. In October of 2005, the resulting young plants were transplanted into 10 cm nursery containers and relocated to an outdoor trial area for field observations. Progeny were further observed for desired commercial characteristics, some were subsequently eliminated and remaining candidates were transplanted into the ground for further observation. In October 2009, ‘WES02’ was selected for its rotund habit, compact size and dense growth. The cultivar was named ‘WES02’.

Asexual Reproduction: ‘WES02’ was first propagated asexually by division in the state of New South Wales, Australia in October of 2009 and has since been asexually propagated by division through six successive generations. The distinctive characteristics of the inventive ‘WESO2’ variety are stable from generation to generation; clones of the variety produced by asexual reproduction maintain the distinguishing characteristics of the original plant.

### SUMMARY OF THE INVENTION

‘WES02’ is a distinctive variety of Westringia fruticosa which is characterized by a combination of a rotund, or ball-shaped, growth habit, short plant height and narrow, fine-textured foliage.

### BRIEF DESCRIPTION OF THE FIGURES

FIG. 1 shows the original ‘WES02’ plant amongst other eliminated candidates in the breeder's trial in July 2011. Of note are the compact size and dense, rotund habit.

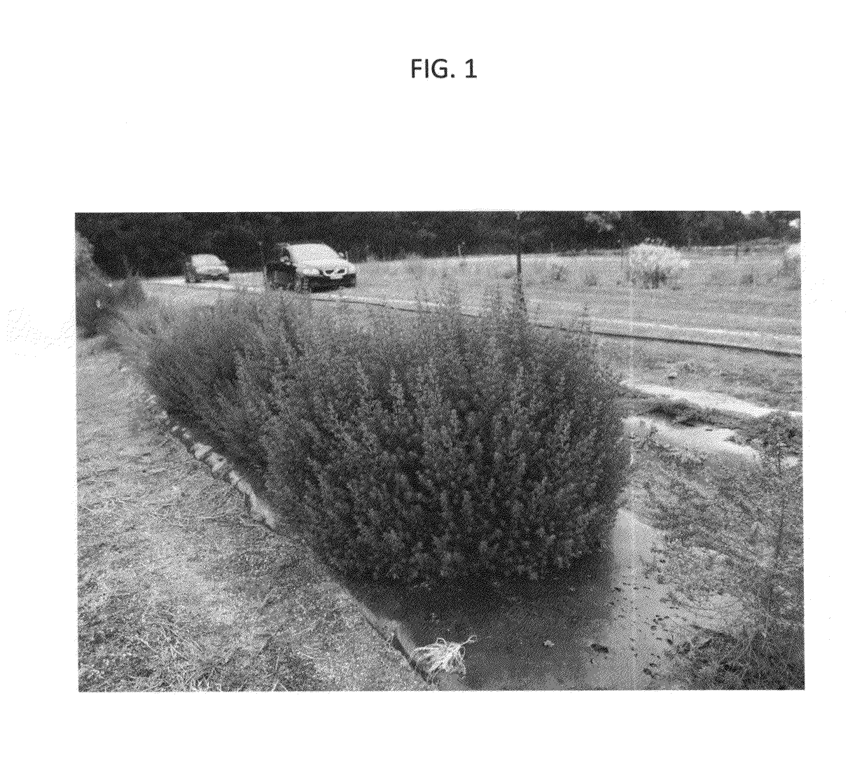

### BOTANICAL DESCRIPTION OF THE PLANT

The following is a detailed botanical description of a new and distinct variety of a Westringia fruticosa ornamental plant known as ‘WES02’. Plant observations were made on plants grown in New South Wales, Australia. Unless indicated otherwise, the descriptions disclosed herein are based upon observations made from mature ‘WES02’ plants grown from rooted cuttings from November 2010 to October 2011 in 200 mm nursery pots filled with soilless potting media, maintained with granular slow release fertilizer and regularly watered with overhead irrigation. No pest and disease measures were taken.

Those skilled in the art will appreciate that certain characteristics will vary with older or, conversely, younger plants. ‘WES02’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 2001 edition. Note that generic color descriptions such as ‘white’ do not exist in the R.H.S. charts and the corresponding R.H.S. colors are quoted.
Technical description of the variety:
Plant description:
   Plant habit.—Shrub; upright, rounded and dense.
   Height.—70 cm at maturity.
   Width.—70 cm at maturity.
   Bloom period.—Spring.
   Hardiness.—USDA Zone 9 to 11.
   Propagation.—Propagation is accomplished using semi-hard wood cuttings. Roots well without rooting compound or hormone.
   Time to develop roots.—approximately 3 weeks.
   Crop time.—From approximately three to four months are needed to produce a well rooted 1-gallon nursery pot, starting from a rooted cutting. Crop time varies with location and production practices.
   Pest and disease susceptibility or resistance.—In common with the species, none of note.
Roots: The roots of ‘WES02’ are colored white and fleshy and thick, similar to other Westringia fruticosa.
Stem:
   Stem strength.—medium.
   Attitude.—acutely branched and upright.
   Shape.—Cylindrical yet angular.
   Stem color.—Young stem is closest to green 137C.
   Stem dimensions.—mature stem, 10 nodes below the actively growing tip, are approximately 19 mm in length with a diameter of 3 mm.
   Stem surface.—Corky and pruinose.
   Basal stem color.—mature stems closest to yellow green 146C.
   Internode length.—12 mm on average.
Foliage:
   Type.—Evergreen.
   Shape.—Ovate to lanceolate.
   Division.—Simple.
   Apex.—Acute.
   Base.—Cuneate.
   Venation.—reticulate but not obvious.
   Vein color (adaxial surfaces).—137A.
   Vein color (abaxial surfaces).—138C.
   Margins.—Entire.
   Arrangement.—Whorled.
   Attachment.—Petiolate.
   Texture.—Leathery.
   Surfaces (adaxial surface).—Finely pubescent to glabrous.
   Surfaces (abaxial surface).—Heavily pubescent.
   Mature leaf dimensions.—average length, 17 mm; width is 5 mm.
   Leaf color (adaxial surface).—137A.
   Leaf color (abaxial surface).—138C.
   Petiole.—Approximately 1 to 2 mm in length; width minute.
   Stipules.—absent.
Inflorescence: None; flowers are sessile.
Bud: Obovoid in shape and rounded at the apex. Approximately 6 mm long and 3 mm wide prior to dehiscence. Color is purple 76A.
Flower:
   General.—Flowers are sessile, simple and persist for approximately 5 days. Self cleaning. Non fragrant.
   Flowers.—Small; Sympetalous, irregular with sepals numbering five, width across the corolla measuring on average 14 mm and a length, from base of perianth to tip of sepal lobe measures 15 mm (average); color approximates to purple 76B. Spots, or “eyes”, present; color closest to 31A.
   Calyx.—Synsepalous with five calyx lobes; yellow green (RHS 146C). The length of the sepal is 6 mm.
Reproductive organs:
   Stamens.—4 and 2 staminodes.
   Stamen color 76D.
   Stamen dimensions.—About 3 mm in length and very thin.
   Attachment.—Adnate.
   Anther dimensions.—Minute; less than 1 mm.
   Anther color.—76A.
   Pollen.—Absent.
   Pistil.—One.
   Pistil dimensions.—About 7 mm in length and very thin.
   Pistil color.—76A.
   Ovary position.—Superior.
Fruit and seed: Observations not yet recorded.
Environmental tolerances:
   Cold and heat tolerance.—The winter hardiness of ‘WES02’ is at least to USDA Zone 9a and evaluation of winter hardiness is ongoing. ‘WES02’ has heat tolerance typical of Westringia fruticosa.
   Drought tolerance.—‘WES02’ has good drought tolerance typical of Westringia fruticosa once established.
   Pest resistance.—No known insect pests. ‘WES02’ has disease tolerance typical of Westringia fruticosa.
   Cultural conditions.—Westringia is commonly known to perform well in sandy soils, but also tolerates heavy, clay-type soils well. (‘WES02’ can also be grown in a relatively wide pH range and is also tolerant of above-normal salt concentration in the soil environment and is also tolerant of aerosol salt exposure; e.g. coastal exposure to salt laden winds.
Comparison of WES02 with Other Varieties of Hybrid Westringia For the purpose of this comparison, the expression of plant characteristics such as “short” and “tall” are employed in accord with the DUS Test Guidelines established under the 1991 Act of the International Union for the Protection of New Varieties of Plants (UPOV) Convention.

When compared to both the seed and pollen parent, ‘WES02’ has a rotund and dense growth habit, a short to medium plant height and medium-width foliage whereas both parents have an upright and openly-branched habit, tall plant height and broad foliage.

‘WES02’ can be compared to two similar varieties of common knowledge, Westringia hybrid ‘WES01’ (unpatented) and Westringia fruticosa ‘Jervis Gem’ (unpatented). However, ‘WES02’ has a short to medium plant height whereas ‘WES01 ’ and ‘Jervis Gem’ have a medium plant height. The flowers of ‘WES02’ are purple (RHS 76B) whereas the flowers of ‘WES01’ are purple violet (86C to 82D). ‘WES02’ has green foliage (RHS137A) with light pubescence whereas the leaves of ‘Jervis Gem’ are yellow green, approximating to RHS 147A, with heavy pubescence which creates a graying effect on the foliage color.

That which is claimed is:

1. A new and distinct variety of Westringia fruticosa plant named ‘WES02’, substantially as described and illustrated herein.
